(19) United States
(12) Patent Application Publication
(10) Pub. No.: US 2026/0248565 A1
(43) Pub. Date: Aug. 27, 2026

(54) VISION-BASED DETECTION OF ACCESS SHEATH

(71) Applicant: Auris Health, Inc., Santa Clara, CA (US)

(72) Inventors: Saif Iftekar Sayed, Foster City, CA (US); John Raymond Young, San Mateo, CA (US); Michael David Leslie, San Francisco, CA (US)

(73) Assignee: Auris Health, Inc., Santa Clara, CA (US)

(21) Appl. No.: 19/060,106

(22) Filed: Feb. 21, 2025

(51) Int. Cl. A61B 34/30; A61B 34/20; G06T 7/10

(52) U.S. Cl. CPC A61B 34/30; A61B 34/20; G06T 7/10; G06T 2207/10068; G06T 2207/20084

(57) ABSTRACT

This disclosure provides methods, devices, and systems for controlling robotically assisted medical systems. The present implementations more specifically relate to techniques for vision-based detection of an access sheath. In some aspects, a controller for a medical system obtains a series of images captured by a camera disposed on a distal tip of a medical instrument that is at least partially inserted through an access sheath. The controller infers a respective segmentation mask from each image in the series based on a machine learning model trained to classify each pixel of the image as depicting the access sheath or not depicting the access sheath, where the segmentation mask indicates how many pixels of the image are classified as depicting the access sheath. The controller further determines a position of the distal tip of the instrument relative to the access sheath based on the segmentation masks for the series of images.

### TECHNICAL FIELD

This disclosure relates generally to medical systems, and specifically to vision-based detection of an access sheath.

### DESCRIPTION OF RELATED ART

Many medical procedures, such as bronchoscopy, laparoscopy, ureteroscopy, or percutaneous nephrolithotomy (PCNL), involve a series of complex steps that require careful movement and positioning of medical tools or instruments inside a patient’s body. The success or failure of such medical procedures often depends on various factors, including the physician’s skill, the patient’s anatomy, and the quality of any tools or equipment the physician uses to perform the procedure. For example, some medical procedures involve the use of shaft-type instruments, such as endoscopes, which may be inserted into the patient and advanced to a target anatomical site. Instrument feeder devices and systems can control the axial movement (such as insertion and retraction) of shaft-type instruments during a medical procedure. The speeds of such axial movements can affect the duration and/or results of the medical procedure. However, instrument insertion and retraction speeds are often governed by system hardware limitations and patient safety considerations.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for determining relative instrument positions. The method includes steps of obtaining a series of images captured by a camera disposed on a distal tip of a medical instrument that is at least partially inserted through an access sheath; inferring a respective segmentation mask from each image in the series of images based on a machine learning model trained to classify each pixel of the image as depicting the access sheath or not depicting the access sheath, where the segmentation mask indicates how many pixels of the image are classified as depicting the access sheath; and determining a position of the distal tip of the medical instrument relative to the access sheath based at least in part on the segmentation masks for the series of images.

Another innovative aspect of the subject matter of this disclosure can be implemented in a controller for a medical system, including a processing system and a memory. The memory stores instructions that, when executed by the processing system, cause the controller to obtain a series of images captured by a camera disposed on a distal tip of a medical instrument that is at least partially inserted through an access sheath; infer a respective segmentation mask from each image in the series of images based on a machine learning model trained to classify each pixel of the image as depicting the access sheath or not depicting the access sheath, where the segmentation mask indicates how many pixels of the image are classified as depicting the access sheath; and determine a position of the distal tip of the medical instrument relative to the access sheath based at least in part on the segmentation masks for the series of images.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIGS. 2 and 3 show example medical system components, according to some implementations.

FIG. 4 shows an example robotic system, according to some implementations.

FIG. 5 shows example retraction speed zones associated with various positions of a medical instrument relative to an access sheath, according to some implementations.

FIG. 6 shows a block diagram of an example system for determining the position of a medical instrument relative to an access sheath, according to some implementations.

FIG. 7 shows a block diagram of an example machine learning system, according to some implementations.

FIG. 8 shows a block diagram of an example instrument position detection system, according to some implementations.

FIG. 9 shows another block diagram of an example instrument position detection system, according to some implementations.

FIG. 10 shows a block diagram of an example controller for a medical system, according to some implementations.

FIG. 11 shows an illustrative flowchart depicting an example operation for determining relative instrument positions, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system’s registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures involve a series of complex steps that require careful movement and positioning of medical tools or instruments inside a patient’s body. Some medical procedures can now be performed, at least in part, by a robotic system or apparatus, which can aid the physician in navigating or positioning such medical instruments. For example, a physician can control a robotic system to advance and navigate a medical instrument (such as a scope) within an anatomy. The robotic system may include, or may be coupled to, one or more display devices that can provide information to assist the physician in navigating the medical instrument. Example suitable information may include real-time images captured by the medical instrument, a visualization to indicate a position and/or orientation (also referred to as a “pose”) of the medical instrument, and/or guidance regarding the procedure, among other examples. Such information can be captured or obtained using various sensors and/or cameras disposed on or otherwise coupled to the medical instrument.

Some medical procedures involve the use of shaft-type instruments, such as endoscopes, which may be inserted into the patient, through an access sheath (also referred to as an “introducer”), and advanced to a target anatomical site. Instrument feeder devices can control the axial movement (such as insertion and retraction) of shaft-type instruments during a medical procedure. The speeds of such axial movements can affect the duration and/or results of the medical procedure. However, instrument insertion and retraction speeds are often governed by system hardware limitations and patient safety considerations. For example, the risk of injury to the patient is high when the distal tip of the instrument interacts with patient anatomy. Thus, the instrument should be inserted and/or retracted slowly and cautiously when the distal tip is outside the access sheath. On the other hand, the risk of injury to the patient is low when the distal tip of the instrument is shielded by the access sheath. Accordingly, the instrument can be inserted and/or retracted faster while the distal tip is disposed within the access sheath. To enable such dynamic control over the speed of instrument movement, the robotic system must know whether the distal tip of the instrument is positioned inside or outside the access sheath.

Some shaft-type instruments, such as endoscopes, have cameras disposed on their distal ends. The camera captures real-time images that can be used for navigating the distal tip within an anatomy (also referred to as “endoscopic vision”). Aspects of the present disclosure recognize that such images can also be used to determine the position of the instrument tip relative to an access sheath. For example, when the instrument tip is positioned inside the access sheath, the inner surface of the sheath can be seen in real-time images captured by the camera disposed on the distal tip. By contrast, when the instrument tip is positioned outside the access sheath, the inner surface of the sheath cannot be seen in the real-time images captured by the camera disposed on the distal tip. Accordingly, a robotic system can determine whether the instrument tip is positioned inside or outside the access sheath based on a presence (or absence) of the access sheath in the images captured by the camera disposed on the distal tip.

In some implementations, a machine learning model may be trained to detect the inner surface of an access sheath in the images captured by a camera disposed on the distal tip of a medical instrument. Machine learning is a technique for improving the ability of a computer system to perform a certain task. Machine learning generally comprises a training phase and an inferencing phase. During the training phase, a machine learning system is provided with one or more “answers” (also referred to as “ground truth”) and a large volume of raw training data associated with the answers. The machine learning system analyzes the training data to learn a set of rules (also referred to as the “machine learning model”) that can be used to describe each of the answers. During the inferencing phase, the machine learning system may infer answers from new data using the learned set of rules.

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. Description of the renal or urinary anatomy and associated medical issues and procedures is presented herein to aid in the description of the concepts disclosed herein. However, it should be understood that these techniques and systems can be implemented in the context of any medical procedure involving an articulable medical instrument (such as an endoscope).

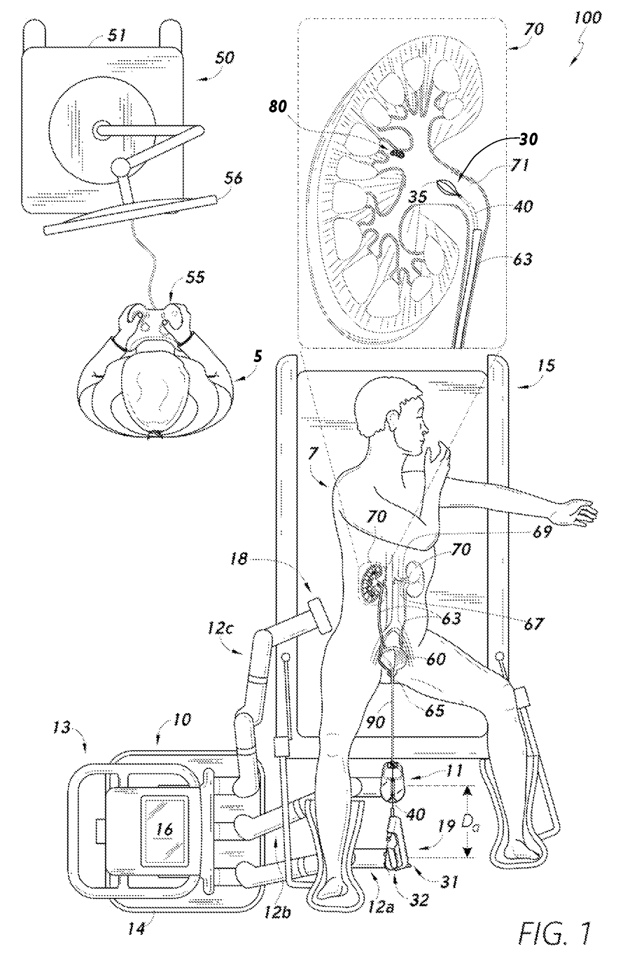

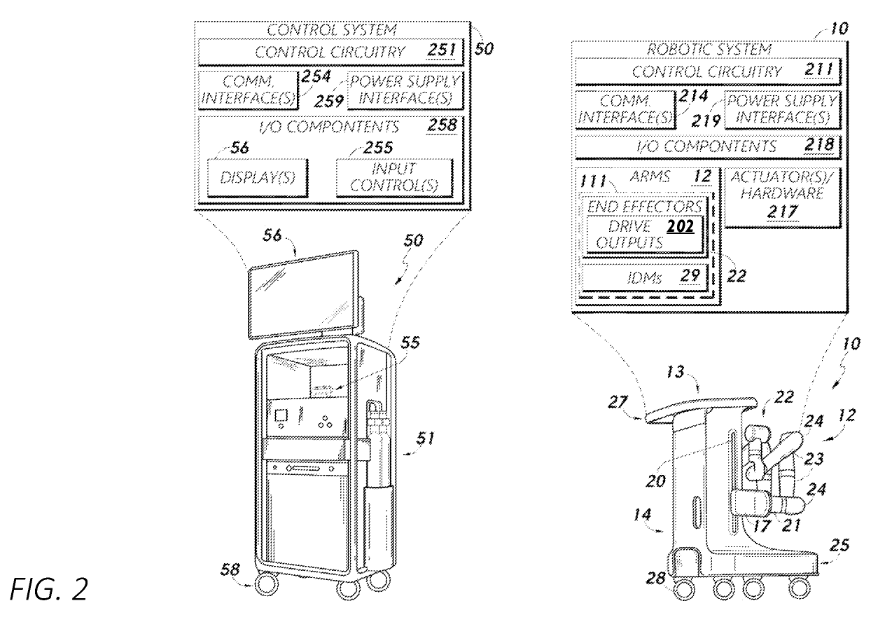

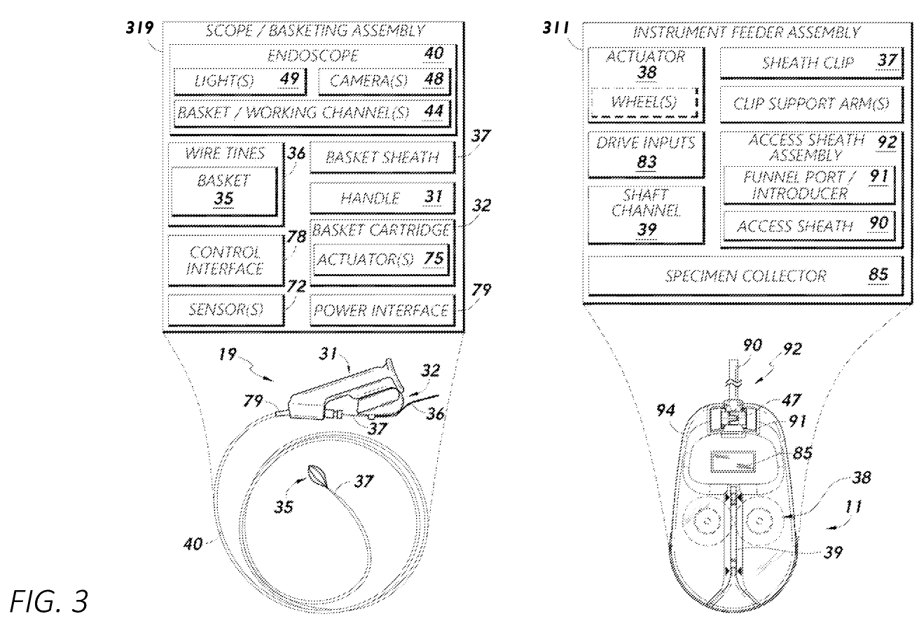

FIG. 1 shows an example medical system 100, according to some implementations. FIGS. 2 and 3 show detailed example implementations of certain components of the medical system 100 shown in FIG. 1. The medical system 100 may be used for, for example, endoscopic (e.g., ureteroscopic) procedures. As referenced and described above, certain ureteroscopic procedures involve the treatment/removal of kidney stones. In some implementations, kidney stone treatment can benefit from the assistance of certain robotic technologies/devices. Robotic medical solutions can provide relatively higher precision, superior control, and/or superior hand-eye coordination with respect to certain instruments compared to strictly-manual procedures. For example, robotic-assisted ureteroscopic access to the kidney in accordance with some procedures can advantageously enable a urologist to individually perform both endoscope control and basketing control.

Although the system 100 of FIG. 1 is presented in the context of a ureteroscopic procedure, it should be understood that the principles disclosed herein may be implemented in any type of endoscopic procedure. Furthermore, several of the examples described herein relate to object removal procedures involving the removal of kidney stones from a kidney. The present disclosure, however, is not limited only to kidney stone removal. For example, the following description is also applicable to other surgical or medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, or cataract removal.

The medical system 100 includes a robotic system 10 (e.g., mobile robotic cart) configured to engage with and/or control a medical instrument 40 (e.g., ureteroscope) to perform a direct-entry procedure on a patient 7. The term “direct-entry” is used herein according to its broad and ordinary meaning and may refer to any entry of instrumentation through a natural or artificial opening in a patient’s body. For example, with reference to FIG. 1, the direct entry of the scope 40 into the urinary tract of the patient 7 may be made via the urethra 65.

The direct-entry instrument 40 can be any type of medical instrument, including an endoscope (such as a ureteroscope), catheter (such as a steerable or non-steerable catheter), nephroscope, laparoscope, or other type of medical instrument. Embodiments of the present disclosure relating to ureteroscopic procedures for removal of kidney stones through a ureteral access sheath (e.g., the ureteral access sheath 90) are also applicable to solutions for removal of objects through percutaneous access, such as through a percutaneous access sheath. For example, instrument(s) may access the kidney percutaneously through, for example, a percutaneous access sheath to capture and remove kidney stones; insertion and retraction speeds of such instruments can be modified/controlled based on instrument position in accordance with aspects of the present disclosure. The term “percutaneous access” is used herein according to its broad and ordinary meaning and may refer to entry, such as by puncture and/or minor incision, of instrumentation through the skin of a patient and any other body layers necessary to reach a target anatomical location associated with a procedure (e.g., the calyx network of the kidney 70).

The medical system 100 includes a control system 50 configured to interface with the robotic system 10, provide information regarding the procedure, and/or perform a variety of other operations. For example, the control system 50 can include one or more display(s) 56 configured to present certain information to assist the physician 5 and/or other technician(s) or individual(s). The medical system 100 can include a table 15 configured to hold the patient 7. The system 100 may further include an electromagnetic (EM) field generator 18, which may be held by one or more of the robotic arms 12 of the robotic system 10 or may be a stand-alone device. Although the various robotic arms are shown in various positions and coupled to various tools/devices, it should be understood that such configurations are shown for convenience and illustration purposes, and such robotic arms may have different configurations over time and/or at different points during a medical procedure. Furthermore, the robotic arms 12 may be coupled to different devices/instruments than shown in FIG. 1, and in some cases or periods of time, one or more of the arms may not be utilized or coupled to a medical instrument (e.g., instrument manipulator/coupling).

In an example use case, if the patient 7 has a kidney stone (or stone fragment) 80 located in the kidney 70, the physician may execute a procedure to remove the stone 80 through the urinary tract (63, 60, 65). In some embodiments, the physician 5 can interact with the control system 50 and/or the robotic system 10 to cause/control the robotic system 10 to advance and navigate the medical instrument 40 (e.g., a scope) from the urethra 65, through the bladder 60, up the ureter 63, and into the renal pelvis 71 and/or calyx network of the kidney 70 where the stone 80 is located. The physician 5 can further interact with the control system 50 and/or the robotic system 10 to cause/control the advancement of a basketing device 30 through a working channel of the instrument 40, wherein the basketing device 30 is configured to facilitate capture and removal of a kidney stone. The control system 50 can provide information via the display(s) 56 that is associated with the medical instrument 40, such as real-time endoscopic images captured therewith, and/or other instruments of the system 100, to assist the physician 5 in navigating/controlling such instrumentation.

The medical instrument 40 (e.g., scope, directly-entry instrument, etc.) can be advanced into the kidney 70 through the urinary tract. Specifically, a ureteral access sheath 90 may be disposed within the urinary tract to an area near the kidney 70. The medical instrument 40 may be passed through the ureteral access sheath 90 to gain access to the internal anatomy of the kidney 70, as shown. Once at the site of the kidney stone 80 (e.g., within a target calyx 75 of the kidney 70 through which the stone 80 is accessible), the medical instrument 40 can be used to channel/direct the basketing device 30 to the target location. Once the stone 80 has been captured in the distal basket portion 35 of the basketing device 30, the utilized ureteral access path may be used to extract the kidney stone 80 from the patient 7.

The various scope-type instruments disclosed herein, such as the scope 40 of the system 100, can be configured to navigate within the human anatomy, such as within a natural orifice or lumen of the human anatomy. The terms “scope” and “endoscope” are used herein according to their broad and ordinary meanings, and may refer to any type of elongate medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), or borescope. Scopes/endoscopes, in some instances, may comprise an at least partially rigid and/or flexible tube, and may be dimensioned to be passed within an outer sheath (also referred to as an “access sheath”), catheter, introducer, or other lumen-type device, or may be used without such devices.

With reference to FIG. 2, the control system 50 can be configured to provide various functionality to assist in performing a medical procedure. In some embodiments, the control system 50 can be coupled to the robotic system 10 and operate in cooperation therewith to perform a medical procedure on the patient 7. For example, the control system 50 can communicate with the robotic system 10 via a wireless or wired connection (e.g., to control the robotic system 10). In some embodiments, the control system 50 can communicate with the EM field generator 18 to control generation of an EM field in an area around the patient 7 and/or around the instrument feeder 11.

Further, in some embodiments, the control system 50 can communicate with the robotic system 10 to receive position data relating to the position of the distal end of the scope 40, access sheath 90, or basketing device 30. Such positional data relating to the position of the scope 40, access sheath 90, or basketing device 30 may be derived using one or more EM sensors associated with the respective components, scope image processing functionality, and/or based at least in part on robotic system data (e.g., arm position data, known parameters or dimensions of the various system components, etc.).

The robotic system 10 can be configured to at least partly facilitate execution of a medical procedure. The robotic system 10 can be arranged in a variety of ways depending on the particular procedure. The robotic system 10 can include one or more robotic arms 12 configured to engage with and/or control, for example, the scope 40 and/or the basketing system 30 to perform one or more aspects of a procedure. As shown, each robotic arm 12 can include multiple arm segments 23 coupled to joints 24, which can provide multiple degrees of movement or freedom. In the example of FIG. 1, the robotic system 10 is positioned proximate to the patient’s legs and the robotic arms 12 are actuated to engage with and position the scope 40 for access into an access opening, such as the urethra 65 of the patient 7. When the robotic system 10 is properly positioned, the scope 40 can be inserted into the patient 7 robotically using the robotic arms 12, manually by the physician 5, or a combination thereof.

A scope-driver instrument coupling 11 (such as an instrument device manipulator (IDM)) can be attached to a distal end effector 22 of one of the arms 12b to facilitate robotic control/advancement of the scope 40. Another 12a of the arms may have associated therewith an instrument coupling/manipulator 19 that is configured to facilitate advancement and operation of the basketing device 30. The instrument coupling 19 may further provide a handle 31 for the scope 40, wherein the scope 40 is physically coupled to the handle 31 at a proximal end of the scope 40. The scope 40 may include one or more working channels through which additional tools, such as lithotripters, basketing devices, forceps, etc., can be introduced into the treatment site.

The robotic system 10 can be coupled to any component of the medical system 100, such as to the control system 50, the table 15, the EM field generator 18, the scope 40, the basketing system 30, and/or any type of percutaneous-access instrument (e.g., needle, catheter, nephroscope, etc.). In some embodiments, the robotic system 10 is communicatively coupled to the control system 50. For example, the robotic system 10 may be configured to receive control signals from the control system 50 to perform certain operations, such as to position one or more of the robotic arms 12 in a particular manner, manipulate the scope 40, and/or manipulate the basketing system 30. In response, the robotic system 10 can control, using certain control circuitry 211, actuators 217, and/or other components of the robotic system 10, a component of the robotic system 10 to perform the operations. For example, the control circuitry 211 may control axial motion of the scope 40 by actuating drive output(s) 202 of the end effector 22 coupled to the instrument feeder 11. In some embodiments, the robotic system 10 and/or control system 50 is configured to receive images and/or image data from the scope 40 representing internal anatomy of the patient 7 and/or portions of the access sheath or other device components.

The robotic system 10 generally includes an elongated support structure 14 (also referred to as a “column”), a robotic system base 25, and a console 13 at the top of the column 14. The column 14 may include one or more arm supports 17 (also referred to as a “carriage”) for supporting the deployment of the one or more robotic arms 12 (three shown in FIG. 1). The arm support 17 may include individually-configurable arm mounts that rotate along a perpendicular axis to adjust the base of the robotic arms 12 for desired positioning relative to the patient.

The arm support 17 may be configured to vertically translate along the column 14. In some embodiments, the arm support 17 can be connected to the column 14 through slots 20 that are positioned on opposite sides of the column 14 to guide the vertical translation of the arm support 17. The slot 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, patient sizes, and physician preferences. Similarly, the individually-configurable arm mounts on the arm support 17 can allow the robotic arm base 21 of robotic arms 12 to be angled in a variety of configurations.

The robotic arms 12 may generally comprise robotic arm bases 21 and end effectors 22, separated by a series of linking arm segments 23 that are connected by a series of joints 24, each joint comprising one or more independent actuators 217. Each actuator may comprise an independently-controllable motor. Each independently-controllable joint 24 can provide or represent an independent degree of freedom available to the robotic arm. In some embodiments, each of the arms 12 has seven joints, and thus provides seven degrees of freedom, including “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The term “end effector” is used herein according to its broad and ordinary meaning and may refer to any type of robotic manipulator device, component, and/or assembly. Where an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. In some contexts, the combination of a robotic end effector and adapter may be referred to as an instrument manipulator assembly, wherein such assembly may or may not also include a medical instrument (or instrument handle/base) physically coupled to the adapter and/or end effector. The terms “robotic manipulator” and “robotic manipulator assembly” are used according to their broad and ordinary meanings, and may refer to a robotic end effector and/or sterile adapter or other adapter component coupled to the end effector, either collectively or individually. For example, “robotic manipulator” or “robotic manipulator assembly” may refer to an IDM including one or more drive outputs, whether embodied in a robotic end effector, sterile adapter, and/or other component(s). The terms “robotic manipulator” and “robotic manipulator assembly” can further refer to a robotic arm or other robotic translator associated with an end effector. The term “end effector,” as used herein, can be understood to refer to any type of robotic manipulator.

The robotic system base 25 balances the weight of the column 14, arm support 17, and arms 12 over the floor. Accordingly, the robotic system base 25 may house certain relatively heavier components, such as electronics, motors, power supply, as well as components that selectively enable movement or immobilize the robotic system. For example, the robotic system base 25 can include wheel-shaped casters 28 that allow for the robotic system to easily move around the operating room prior to a procedure. After reaching the appropriate position, the casters 28 may be immobilized using wheel locks to hold the robotic system 10 in place during the procedure.

Positioned at the upper end of column 14, the console 13 can provide both a user interface for receiving user input and a display screen 16 (or a dual-purpose device such as, for example, a touchscreen) to provide the physician/user with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 13 may be positioned and tilted to allow a physician to access the console from the side of the column 14 opposite arm support 17. From this position, the physician may view the console 13, robotic arms 12, and patient while operating the console 13 from behind the robotic system 10. As shown, the console 13 can also include a handle 27 to assist with maneuvering and stabilizing the robotic system 10.

The end effector 22 of each of the robotic arms 12 may comprise, or be configured to have coupled thereto, an instrument device manipulator (IDM) 29, which may be attached using a sterile adapter component in some instances. The combination of the end effector 22 and associated IDM, as well as any intervening mechanics or couplings (e.g., sterile adapter), can be referred to as a manipulator assembly 111. In some embodiments, the IDM 29 can be removed and replaced with a different type of IDM, for example, a first type 11 of IDM may be configured to manipulate an endoscope, while a second type 19 of IDM may manipulate a basketing device and/or support a proximal end of the endoscope. Another type of IDM may be configured to hold an electromagnetic field generator 18. An IDM can provide power and control interfaces. For example, the interfaces can include connectors to transfer pneumatic pressure, electrical power, electrical signals, and/or optical signals from the robotic arm 12 to the IDM. The IDMs 29 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the device manipulators 29 can be attached to respective ones of the robotic arms 12, wherein the robotic arms 12 are configured to insert or retract the respective coupled medical instruments into or out of the treatment site.

As referenced above, the medical system 100 can include certain control circuitry configured to perform certain of the functionality described herein, including the control circuitry 211 of the robotic system 10 and the control circuitry 251 of the control system 50. That is, the control circuitry of the medical system 100 may be part of the robotic system 10, the control system 50, or some combination thereof. Therefore, any reference herein to control circuitry may refer to circuitry embodied in a robotic system, a control system, or any other component of a medical system, such as the medical system 100 of FIG. 1. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions.

Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211/251 may be entirely locally maintained/disposed or may be remotely located at least in part (e.g., communicatively coupled indirectly via a local area network and/or a wide area network).

With respect to the robotic system 10, at least a portion of the control circuitry 211 may be integrated with the base 25, column 14, and/or console 13 of the robotic system 10, and/or another system communicatively coupled to the robotic system 10. With respect to the control system 50, at least a portion of the control circuitry 251 may be integrated with the console base 51 and/or display unit 56 of the control system 50. Any description herein of functional control circuitry or associated functionality may be understood to be embodied in the robotic system 10, the control system 50, or any combination thereof, and/or at least in part in one or more other local or remote systems/devices.

With further reference to FIG. 2, the control system 50 can include various I/O components 258 configured to assist the physician 5 or others in performing a medical procedure. For example, the input/output (I/O) components 258 can be configured to allow for user input to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or catheter basketing system 30.

The control system 50 can include one or more display devices 56 to provide various information regarding a procedure. For example, the display(s) 56 can provide information regarding the scope 40 and/or basketing system 30. For example, the control system 50 can receive real-time images that are captured by the scope 40 and display the real-time images via the display(s) 56. Additionally or alternatively, the control system 50 can receive signals (e.g., analog, digital, electrical, acoustic/sonic, pneumatic, tactile, hydraulic, etc.) from a medical monitor and/or a sensor associated with the patient 7, and the display(s) 56 can present information regarding the health or environment of the patient 7. Such information can include information that is displayed via a medical monitor including, for example, information relating to heart rate (e.g., ECG, HRV, etc.), blood pressure/rate, muscle bio-signals (e.g., EMG), body temperature, blood oxygen saturation (e.g., SpO₂), CO₂, brainwaves (e.g., EEG), or environmental and/or local or core body temperature.

To facilitate the functionality of the control system 50, the control system can include various components (sometimes referred to as “subsystems”). For example, the control system 50 can include the control electronics/circuitry 251, as well as one or more power supplies/supply interfaces 259, pneumatic devices, optical sources, actuators, data storage devices, and/or communication interfaces 254. In some embodiments, the control system 50 is movable, while in other embodiments, the control system 50 is a substantially stationary system. Although various functionality and components are discussed as being implemented by the control system 50, any of such functionality and/or components can be integrated into and/or performed by other systems and/or devices, such as the robotic system 10, the basketing system 30, the table 15, and/or others, for example.

With further reference to FIG. 1, the medical system 100 can provide a variety of benefits, such as providing guidance to assist a physician in performing a procedure (e.g., instrument tracking, instrument alignment information, etc.), enabling a physician to perform a procedure from an ergonomic position without the need for awkward arm motions and/or positions, enabling a single physician to perform a procedure with one or more medical instruments, avoiding radiation exposure (e.g., associated with fluoroscopy techniques), enabling a procedure to be performed in a single operative setting, or providing continuous suction to remove an object more efficiently (e.g., to remove a kidney stone). For example, the medical system 100 can provide guidance information to assist a physician in using various medical instruments to access a target anatomical feature while minimizing bleeding and/or damage to anatomy (e.g., critical organs, blood vessels, etc.).

Further, the medical system 100 can provide non-radiation-based navigational and/or localization techniques to reduce physician and patient exposure to radiation and/or reduce the amount of equipment in the operating room. Moreover, the medical system 100 can provide functionality that is distributed between the control system 50 and the robotic system 10, which may be independently movable. Such distribution of functionality and/or mobility can enable the control system 50 and/or the robotic system 10 to be placed at locations that are optimal for a particular medical procedure, which can maximize working area around the patient 7 and/or provide an optimized location for the physician 5 to perform a procedure.

The various components of the system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the various communication interfaces of the systems of FIG. 2 can be configured to communicate with one or more device/sensors/systems, such as over a wireless and/or wired network connection. In some embodiments, the various communication interfaces can implement a wireless technology such as Bluetooth, Wi-Fi, near-field communication (NFC), or the like. Furthermore, in some embodiments, the various components of the system 100 can be connected for data communication, fluid exchange, and/or power exchange via one or more support cables or tubes.

The control system 50, basketing system 30 (see FIG. 3), and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to respective control circuitry.

In some embodiments, a user can manually manipulate a robotic arm 12 of the robotic system 10 without using electronic user controls. For example, during setup in a surgical operating room, a user may move the robotic arms 12 and/or any other medical instruments to provide desired access to a patient. The robotic system 10 may rely on force feedback and inertia control from the user to determine appropriate configuration of the robotic arms 12 and associated instrumentation.

FIG. 3 shows a scope and/or basketing assembly 319 and an instrument feeder assembly 311 that may be implemented in the medical system 100 of FIG. 1, according to some implementations. The scope/basket system 319 comprises various hardware and control components. In some embodiments, the scope/basket system 319 includes handle 31 coupled to an endoscope 40. For example, the scope can include an elongate shaft including one or more lights 49 and one or more cameras or other imaging devices 48. The scope 40 can further include one or more working channels 44, which may run a length of the scope 40. In some embodiments, such channel(s) may be utilized to provide access for elongate basketing wires/tines through the scope 40.

The scope/basket system 319 can comprise a basket 35 formed of one or more wire tines 36. For example, the basketing system 30 may comprise four wire tines disposed within a basketing sheath 37 over a length thereof, wherein the tines project from a distal end of the sheath 37 to form the basket form 35. The tines 36 further extend from the proximal end of the sheath 37. The tines 36 may be configured to be slidable within the basketing sheath 37, subject to some amount of frictional resistance. The tines 36 and the sheath 37 can be coupled to respective actuators 75 of a basket cartridge component 32. The basket cartridge 32 may be physically and/or communicatively coupled to the handle portion/component 31 of the scope/basket system 319. The handle component 31 can be configured to be used to assist in basketing and/or scope control either manually or through robotic control.

The scope/basket system 319 can be powered through a power interface 79 and/or controlled through a control interface 78, each or both of which may interface with a robotic arm/component of the robotic system 10. The scope/basket system 319 may further comprise one or more sensors 72, such as pressure and/or other force-reading sensors, which may be configured to generate signals indicating forces experienced at/by one or more of the actuators 75 and/or other couplings of the scope/basket system 319. Such sensor readings may be used to determine stuck basket conditions, as described in detail herein.

FIG. 3 further illustrates an instrument feeder/driver assembly 311 including an instrument feeder/driver 11 and an access sheath assembly 92, which may be physically coupled to the instrument feeder 11. The terms “feeder” and “driver” are used in some contexts herein substantially interchangeably. Therefore, references herein to a scope or instrument feeder can be understood to refer to any type of scope or instrument driver, and vice versa, wherein such devices/systems are configured to actuate, or cause actuation of, a shaft-type instrument in an axial dimension. The instrument feeder assembly 311 can include a channel 39 dimensioned and/or configured for placement therein of at least a portion of a shaft-type instrument, such as an endoscope or the like. For example, when placing a scope or the like to allow for the instrument feeder 11 to axially drive such instrument, the instrument may be nested at least partially within the channel 39. Although illustrated with a channel 39, in some embodiments, instrument feeder devices and assemblies in accordance with aspects of the present disclosure may not include such a channel.

The actuator 38 may comprise a feed-roller in some embodiments. As used herein, the term “feed-roller” may include any number of roller(s)/wheel(s) configured to effect axial movement of a shaft engaged therewith. “Feed-roller” may further include the shaft channel 39, as well as any input or output drives associated with the instrument feeder 11 that cause, directly or indirectly, movement of the roller(s)/wheel(s).

In some embodiments, the access sheath 92 is not docked to the instrument feeder 11, but rather coupled to a robot arm, a stand, or other structure. Although certain embodiments described herein refer to access sheath assemblies including port/introducer structure and sheath components, it should be understood that embodiments of the present disclosure may implement access sheaths that have integrated port and sheath components. Therefore, references herein to an “access sheath,” or simply “sheath,” may refer to a sheath portion, port portion, or both, of an access sheath/assembly. That is, references herein to any component or portion of an access sheath assembly can be understood to refer to a sheath portion/component, a port/introducer portion/component, or both. Furthermore, access sheath assemblies described herein may be a unitary device, form, or structure, rather than an assembly of separate components.

The instrument feeder assembly 311 further includes an axial actuator means or mechanism 38, which may comprise one or more shaft-engagement wheels, conveyor belts, gears, tracks, or other actuator(s). The actuator 38 is configured to cause a shaft-type instrument placed in engagement therewith to be moved with respect to an axis of the instrument. The actuator(s) 38 can be controlled through engagement with one or more drive inputs 83, which may allow for physical engagement with mechanical components of the instrument feeder 11 that actuate the actuator means/mechanism 38 and/or may directly actuate the actuator means/mechanism 38.

The instrument feeder assembly 311 further includes a sheath clip 47, which may be associated with the instrument feeder 11 and configured to secure or hold in place at least a portion of the access sheath assembly 92. For example, the clip 47 may be configured to clamp on or over at least a portion of a funnel port structure 91 of the access sheath assembly 92, as shown. The access sheath assembly 92 includes an access sheath tube or conduit 90, which may be physically coupled at a proximal end thereof to the funnel port structure 91, which may provide an at least partially conical introducer opening into the access sheath 90, wherein a proximal opening of the port 91 has an area or diameter greater than the cross-sectional area or diameter of the access sheath 90. The clip 47 may be supported by one or more clip support arms 94.

In some embodiments, the instrument feeder assembly 311 includes a specimen collector structure 85, which may be secured at least in part to one or more components of the instrument feeder assembly 311 and/or instrument feeder 11. The specimen collector 85 may comprise a cup-like or other structure configured to allow for placement or dropping therein of a kidney stone or other specimen or debris retracted through the access sheath assembly 92. In some embodiments, the specimen collector 85 is disposed between the distal opening of the channel 39 and the funnel port structure 91, wherein the instrument may be retracted to a position over the specimen collector such that the stone/specimen may be dropped or placed in the specimen collector 85.

The system 100 may advantageously be configured to implement certain scope retraction speed control or modification based on determined or detected scope position as disclosed in detail herein. Such scope speed control may advantageously provide for efficient stone removal and prevention or reduced risk of damage do tissue and/or instrumentation during scope insertion and retraction. For example, the medical system 100 may limit or reduce the speed of instrument insertion and/or retraction when the distal tip of the instrument is positioned outside the access sheath (e.g., beyond the distal opening). On the other hand, the medical system 100 may allow for faster instrument insertion and/or retraction when the distal tip of the instrument is positioned inside the access sheath (e.g., beneath the distal opening).

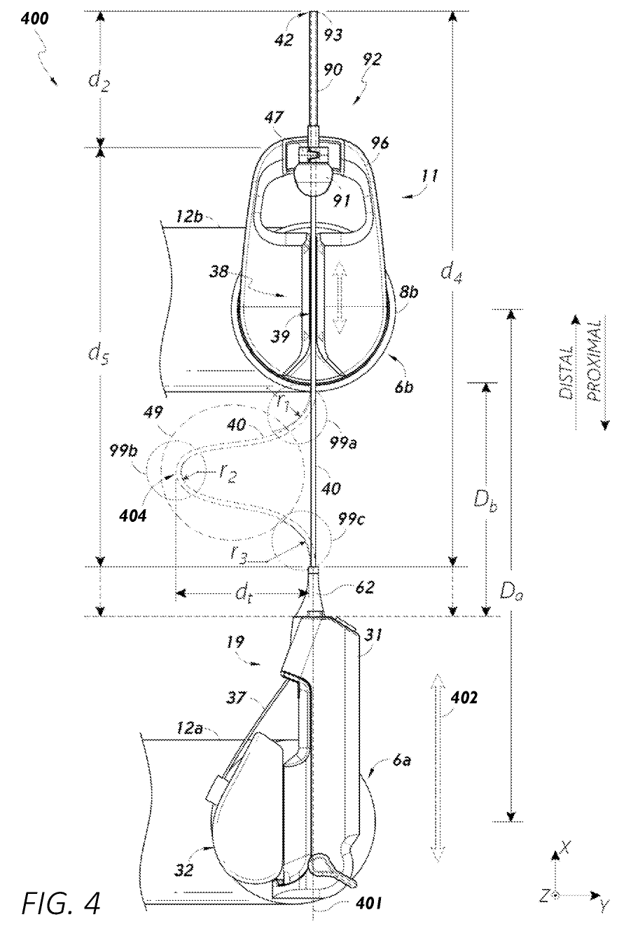

FIG. 4 shows an example robotic system 400, according to some implementations. FIG. 4 shows a robotic system 400 including a first medical instrument 19 (e.g., endoscope, ureteroscope, or the like) including an elongate shaft 40 associated with a first robotic arm 12a. In the description of FIG. 4, as with any other embodiment disclosed herein, robotic arms are described for convenience; it should be understood that description of robotic arms and end effectors associated with the distal end of a robotic arm can be any type of robotic manipulator (e.g., end effector) capable of translation in space, such as along an insertion or retraction path or rail. Therefore, references herein to a robotic arm can be understood to refer to any type of robotic manipulator, such as any type of robotic insertion mechanism, linear actuator/translator, rail drive, or the like.

The instrument 19 may include a handle 31, which may be attached or mounted to an end effector 6a and/or adapter component 8a associated with the robot arm 12a. The system 400 further includes an instrument feeder device 11 configured to axially retract and/or insert the elongate shaft 40 of the instrument 19 when configured as shown in FIG. 4. The instrument feeder 11 is associated with a second robotic arm 12b. For example, the instrument feeder 11 may be attached or mounted to an end effector 6b and/or adapter component 8b associated with the robot arm 12b, as described and illustrated in the present disclosure.

The system 400 can be configured to retract and insert the elongate shaft 40 through and/or at least partially within a sheath 90 of an access sheath assembly 92. The access sheath assembly 92 may include an introducer port 91, which may be secured to a clip 47 or other feature of the instrument feeder 11. In order to effect such insertion and/or retraction, the actuator means/mechanism 38 of the instrument feeder 11 (e.g., feed-roller wheel(s), track, belt, or the like) can axially move the elongate shaft 40 relative to the feeder device 11. Furthermore, insertion and/or retraction can be facilitated by the movement of the robot arm 12a and/or end effector 6a in a direction parallel with the axis 401 of the sheath 90 and/or at least a portion of the elongate shaft 40, as indicated in FIG. 4. For example, when retracting the elongate shaft 40, the axial actuator means/mechanism 38 of the feeder device 11 may cause retraction of the shaft 40 in the proximal direction.

In addition, or as an alternative, the robot arm 12a may be actuated to move the end effector 6a in the proximal direction along the axis/rail 401 to withdraw at least a portion of the shaft 40 proximately, thereby reducing a service loop 49 in the shaft 40 between the feeder 11 and handle 31 that might otherwise form if the feeder 11 retracts the shaft 40 without increasing the distance D a between the robot arms 12a and 12b. During instrument insertion, the robot arm 12a can be moved distally toward the feeder 11 to avoid running out of slack in the service loop 49 when the instrument feeder 11 inserts the shaft 40, as may occur if insertion is implemented by the instrument feeder without any increase in distance D a between the robot arms 12a, 12b. In the event that slack in the service loop 49 is completely exhausted during insertion and the distance D a is not decreased accordingly, the absence of slack may limit the ability of the feeder device 11 to further insert the shaft 40. Furthermore, damage can be caused to the shaft 40, actuator means/mechanism 38, instrument 19, robotic arm (s) 12 a/12b, and/or other instrumentation when further insertion is attempted without any available slack in the shaft 40 between the instrument feeder 11 and the instrument handle 31.

If the instrument feeder 11 (e.g., scope driver) and instrument end effector 6a are operated at the same speed throughout a stone insertion or retraction process, the process may be undesirably slow due to the relatively limited speeds of operation for end effector translation relative to the operating speeds of the shaft actuator(s) of the feeder. Such translation speed limitations can be due to safety and/or damage concerns with respect to the patient and/or instrumentation, or may be based on other physical and/or environmental constraints; fast robotic end effector movement (e.g., for the instrument arm 12a) can be perceived as relatively risky and can cause collision with other objects. At the same time, large retraction distances/lengths can require relatively large workspaces for arm/end effector motion which can be hard to achieve.

In view of such considerations, driving the feeder actuator(s) and the scope/instrument end effector 6a at different speeds during one or more stages of an insertion or retraction process can advantageously make the procedure more efficient, safe, and/or tenable. With respect to stone extraction and/or retraction processes, driving the feeder actuator(s) 38 at relatively high speed during stone extraction can improve efficiency. However, without commensurate increase in the distance D a between the robot arms from translating the end effector 6a (and/or end effector 6b) in space, a relatively large curvature can form in the service loop 49 between the instrument/scope feeder 11 and the instrument end effector 6a, which can cause scope damage. Thus, the coordination between the feeder operation and the instrument handle translation (e.g., via translation of the robotic end effector 6a) during stone extraction (e.g., insertion and/or retraction) can be important for providing improved efficiency and avoiding damage to the instrument in connection with the various embodiments disclosed herein.

In some systems, it may be necessary or desirable to limit the proximal or distal speed and/or distance of movement/translation of the robot arm 12a and/or end effector 6a based on workspace limitations. For example, the arm 12a may have a limited range of motion within the physical parameters of the robotic system. That is, the arm 12a and/or end effector 6a may allow for only a limited range of movement along the rail 401, which may be a virtual rail along which the end effector 6a is configured to be actuated to keep the shaft 40 substantially axial and/or in-line with the channel 39 of the feeder device 11 and/or the access sheath 90. Furthermore, the speed of movement of the robot arm 12a may be limited by mechanical constraints and/or as a means of maintaining safe operation of the robotic system. For example, exactness in movement and/or position may be compromised when the robot arm 12a and/or end effector 6a are translated at too high of speeds.

Generally, the axial actuator means/mechanism 38 of the instrument driver 11 may be configured to axially move (e.g., insert and/or retract) the elongate shaft 40 at a speed that is greater than the maximum distal and/or proximal translation speed of the robot arm 12a, end effector 6a, and/or instrument handle 31. It may be desirable to insert and/or retract the shaft 40 at such relatively high speeds during certain portions of a retraction and/or insertion process in order to provide desirable and/or improved efficiency for execution of the procedure. That is, it may not be desirable to limit the axial actuation of the shaft 40 by the instrument feeder 11 to the maximum retraction and/or insertion speed of the robot arm 12a associated with instrument handle 31. Therefore, the processes disclosed herein may involve maintaining and/or utilizing certain service loop configurations and/or conditions during insertion and/or retraction as a means of allowing for the relatively high retraction/insertion speeds of the instrument feeder 11 to be utilized to quickly insert and/or retract the elongate shaft 40.

Certain embodiments are disclosed herein relating to insertion and/or retraction of elongate shafts of certain medical/surgical instruments, wherein such insertion/retraction is implemented using an instrument feeder device and/or actuator component(s) thereof. However, it should be understood that any description herein of insertion or retraction of an elongate shaft or other instrument may be achieved/performed using instrument feeder actuation and/or robot arm/end-effector translation, as shown in the example implementation in FIG. 4. Furthermore, although FIG. 4 and other figures of the present disclosure show instrument handles and drivers/feeders attached to end effectors associated with distal ends of robotic arms, it should be understood that robotic end effector translation in connection with instrument insertion and/or retraction processes/functionality may be implemented using any type of end effector, whether associated with a robotic arm or not. For example, some systems may include robotic end effectors disposed on a track or other structure, wherein translation of such end effectors can be achieved by sliding/running along the track or other structure.

As a means of promoting safety and efficiency in connection with the various embodiments of the present disclosure, the instrument shaft 40 may advantageously be retracted or inserted at different speeds depending on determination of a present position of the distal end of the shaft 40 according to any of the position determination means/mechanisms disclosed herein. For example, the position of the shaft tip 42 relative to the tip 93 of the access sheath 90 may be used to govern speed of operation of the feeder actuator(s) 38 and/or end effector 6a translation. Although end effector translation (e.g., for an end effector associated with a medical instrument, such as an endoscope) is disclosed herein as generally being along a rail that is in-line with the feeder channel and/or sheath axis, such translation need not be along such rail, and rather may be along a path that is angled with respect to such reference lines. Furthermore, up and down translation may be implemented in connection with the various processes disclosed herein to achieve the desired distances between end effectors/instruments. In addition, plates or other components of an end effector or end effector adapter may be rotated to provide the desired distances for shaft service loops and/or reduce the curvature present in such service loops.

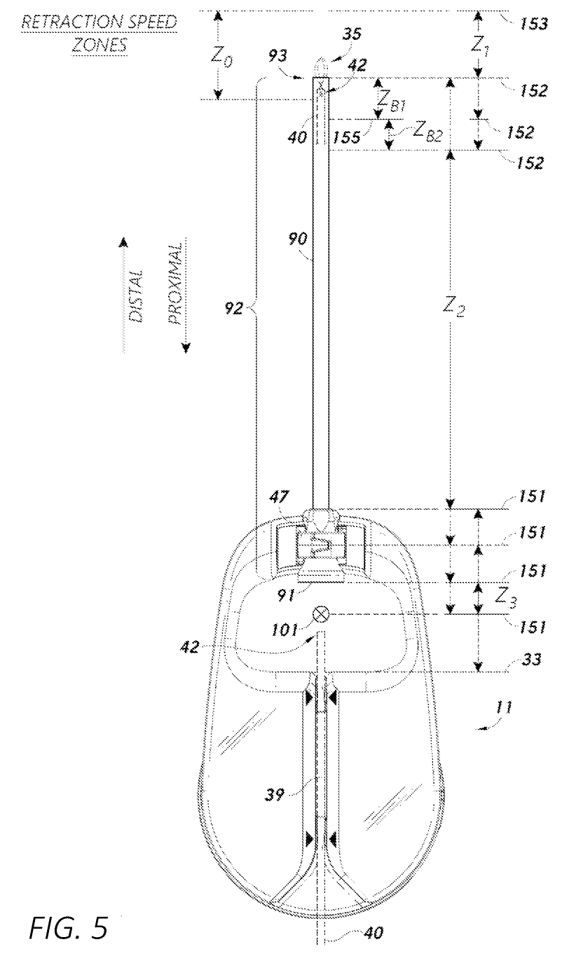

FIG. 5 shows an assembly of an instrument driver/feeder device 11 and an access sheath assembly 92, wherein certain scope retraction speed zones are identified in accordance with one or more embodiments. With respect to processes for retracting a scope 40 into and through an access sheath assembly 92, such as may be implemented after a stone fragment has been collected following insertion, the insertion and retraction process(es) can advantageously be repeated multiple times in a single surgical setting. Retraction of the scope 40 into the sheath 90 of the access sheath assembly 92 can require the physician to pay close attention and/or operate the retraction at relatively low speeds for the purpose of avoiding damage to the patient, the scope assembly 92, and/or other instrumentation that can result from overly aggressive retraction outside the distal end 93 of the sheath.

According to some position-based retraction speed control schemes of the present disclosure, the total travel path of the distal end of the relevant instrument shaft 40 can be divided into two, three, four, or more different zones, wherein the speed of retraction may be executed/determined differently based on which zone the shaft tip is presently in. For example, such zones may include one or more of slow retract zones Z 1, Z 3, normal/fast-retract-buffer zones Z B1, Z B2, fast retract zones Z 2, and/or pause/stop zones/locations, wherein the retraction speeds implemented in the respective zones may be implemented automatically.

Initially, when retracting the distal end of the scope or other elongate shaft instrument 40 into the access sheath 90, retraction involves bringing the distal end 42 of the scope into the distal opening 93 of the access sheath 90. According to the scheme of FIG. 5, the area immediately distal to the access sheath 90 may be within a slow retract zone Z 1. The system control circuitry may be configured to control retraction speed of the shaft 40 at a relatively slow speed to allow for retraction of the distal end 42 of the shaft 40 into the distal end 93 of the access sheath 90 and provide confirmation input confirming the successful entry of the distal end 42 of the shaft and/or the basket 35 or other working instrument associated therewith. Control of retraction speed of the shaft 40, as implemented using any control circuitry of the system, may be based on presence/position determination of the tip 42 of the shaft 40 within any of the various zones shown in FIG. 5.

After the scope tip 42 has been retracted into the sheath tip 93, the scope tip 42 may enter a ‘normal,’ or ‘buffer,’ retract zone (Z B1and/or Z B2; also referred to as ‘fast retract buffer zone(s)’ in some contexts for convenience and/or clarity). The fast retract buffer zone(s) can be implemented to ensure that a basket tip 35 (or other working instrument) protruding from the shaft 40 is also safely retracted into the sheath 90 without becoming stuck. As an additional consideration, the distance between the robotic end effector (e.g., robot arm end effector) associated with the instrument handle and the end effector associated with the instrument feeder 11 may also need to be greater than a certain threshold distance when fast retraction is initiated to ensure a curvature associated with a service loop of the shaft is not too tight, which could cause damage to the instrument shaft 40. As a result, depending on the length d 2 of the access sheath 90, fast retraction may be initiated once the shaft tip 42 passes the threshold 152 associated with the distal boundary of the fast retract zone Z 2.

In some implementations, an additional fast retract buffer zone Z B2 may exist between the initial fast retract buffer zone Z B1 and the fast retract zone Z 2 where the shaft retract speed is driven at relatively higher speeds than in the first buffer zone Z B1 but lower speed than the fast retract zone in order to increase the distance between the feeder and the instrument handle until it meets the minimum distance required to start fast retraction. After the scope tip 42 retracts into the fast retract zone (Z 2), the instrument feeder actuator(s) can be accelerated to a maximum operating speed. In some embodiments, once fast retraction has been initiated, the instrument shaft may be retracted at the fast retraction speed until an automatic pause position 101 is reached. During the retraction process, if the instrument handle end effector reaches a retraction workspace limit, the retraction translation of the instrument end effector may be paused, such that retraction is achieved solely through axial actuation of the shaft 40 by the instrument feeder 11.

In some implementations, the sheath-entry confirmation zone Z 0 may overlap with the slow retract zone Z 1, which may span an area distal to the access sheath 90 as well as a distal portion of the access sheath 90. In some implementations, the slow retract zone Z 1 only includes an area distal to the distal end 93 of the access sheath 90, whereas the distal end 93 of the access sheath 90 represents a threshold transition into another more proximal zone. Within the slow retract zone Z 1, retraction speed may be limited to a relatively slow speed compared to other retraction speeds implemented in connection with the retraction scheme associated with FIG. 5 and/or any other embodiment of the present disclosure.

According to some retraction (and/or insertion) schemes, control of retraction (or insertion) may be implemented in connection with a plurality of speeds, including, for example, a slow insertion/retraction speed may represent a slowest speed or speed limit of the relevant retraction/insertion scheme. A ‘normal’ insertion/retraction speed may represent a speed of retraction/insertion that is greater than the slow speed, and may represent a default retraction/insertion speed, or other speed typical of certain procedural stages of an insertion or retraction process. An ‘intermediate’ speed may represent a speed of retraction/insertion that is greater than the normal speed. A ‘fast’ insertion/retraction speed may represent a speed of retraction/assertion that is greater than the intermediate speed and may represent a maximum possible or allowable retraction/insertion speed. For example, such fast retraction/insertion speeds may be implemented strictly within an access sheath to avoid damage to instrumentation and/or patient anatomy.

According to the retraction speed zone scheme of FIG. 5, a portion of the slow retract zone Z 1 may be considered a fast retract buffer zone Z B1 within the access sheath 90. For example, position determination of the distal end 42 of the shaft 40 between the distal end 93 of the access sheath 90 and a threshold 155 a certain distance from the distal end 93 of the access sheath may indicate that the shaft 40 is within the access sheath 90, but not a far enough distance within the access sheath 90 to ensure that the basket 35 or other working instrument associated with the shaft 40 has also been brought within the access sheath 90. In some embodiments, the threshold 155 associated with the proximal boundary of the fast retract buffer zones Z B1 may correspond to a threshold 152 associated with the proximal end of the slow retract zone Z 1. In some implementations, the retraction speed within the slow retract zone Z 1 and/or fast retract buffer zone Z B1 may be limited to a normal retraction speed.

An area within the sheath 90 that is proximal to the slow retract zone Z 1 and/or fast retract buffer zone Z B1 one may be considered a fast retract zone Z 2, in which retraction speed may be increased to a relatively fast speed, which may represent a maximum retraction speed for the system. In some embodiments, a zone Z B2 may be present between the proximal threshold 152 of the slow retract zone Z 1 and the distal threshold of the fast retract zone Z 2. In such embodiments, retraction in the zone Z B2 may be implemented at an intermediate speed that is faster than the normal speed but less than the fast retraction speed. For example, the intermediate may correspond to a maximum robotic translation speed associated with the robotic end effector attached to the handle or base of the instrument being retracted.

In some embodiments, the fast retract zone Z 2 may extend proximately past a proximal end of the sheath 90 and/or introducer component 91 of the access sheath 92. For example, the fast retract zone Z 2 may extend to the automatic pause/stop position 101, as described in detail herein. For example, the system control circuitry may be configured to implement fast retraction through the proximal end of the access sheath assembly 92 and to automatically stop/pause at the location 101. In some embodiments, a slow retract zone Z 3 may be implemented between a proximal portion of the access sheath assembly 92 and the automatic pause location 101, such that retraction of the scope may proceed at the maximum speed through the fast retract zone Z 2, but slow down to a relatively slower speed (e.g., intermediate, normal, or slow speed, as defined above) prior to ultimately stopping/pausing at the automatic pause location 101.

As described with reference to FIGS. 4 and 5, the robotic system 400 can dynamically vary the speed of insertion and retraction of the shaft 40 based on the position of the distal tip 42 relative to the distal opening 93 of the access sheath 90. In some implementations, the relative instrument positions and/or retraction/insertion speed can be determined and/or controlled based on system data input by a user. For example, data relating to the scope length d 4, sheath length d 2, the distance D a between the scope arm end effector 6a and the scope feeder end effector 6b can be obtained from the system and/or input by a user. Such information can be used to determine or calculate the position of the distal end 93 of the sheath 90 with respect to the position of the distal end of the shaft 40. However, errors and/or inaccuracies in the information provided (such as due to mechanical slippage, improper sheath clipping, standard deviation error in manufacturing length, and/or incomplete elimination of scope slack) can lead to inaccurate calculation of the relative position of the distal tip 42.

According to some implementations, a sheath entry confirmation zone Z 0 represents an area distal to the distal end 93 of the sheath 90 and covering a distal portion of the sheath 90 as well, in which area the operator/technician may provide an indication of confirmed successful entry into the access sheath 90 once the distal tip 42 of the shaft 40 has successfully entered the access sheath 90. While the shaft 40 is in the confirmation zone Z 0, a pop-up window or other graphical interface may be generated and/or presented to the operator/technician to confirm the position of the distal end 93 of the access sheath 90. For example, to ensure safety, fast retraction mode may only be enabled/permitted after the user has confirmed the sheath tip 93 position (e.g., as visible on a camera image of the instrument camera). A user should confirm the sheath tip 93 position when the distal tip 42 is within a threshold distance from the sheath tip 93. However, the point at which such confirmation is provided may vary among different users.

Aspects of the present disclosure recognize that the process for confirming the sheath tip 93 can be automated, at least in part, by a machine learning model trained to detect the inner surface of the access sheath 90 in images captured by a camera disposed on the distal tip 42 of the shaft 40 (such as images used for endoscopic vision). For example, when the distal tip 42 is positioned inside the access sheath 90, the inner surface of the sheath 90 can be seen in real-time images captured by the camera. By contrast, when the distal tip 42 is positioned outside the access sheath 90, the inner surface of the sheath 90 cannot be seen in the real-time images captured by the camera. Accordingly, the robotic system 400 can determine whether the distal tip 42 is positioned inside or outside the access sheath 90 based on a presence (or absence) of the inner surface of the access sheath 90 in the images captured by the camera.

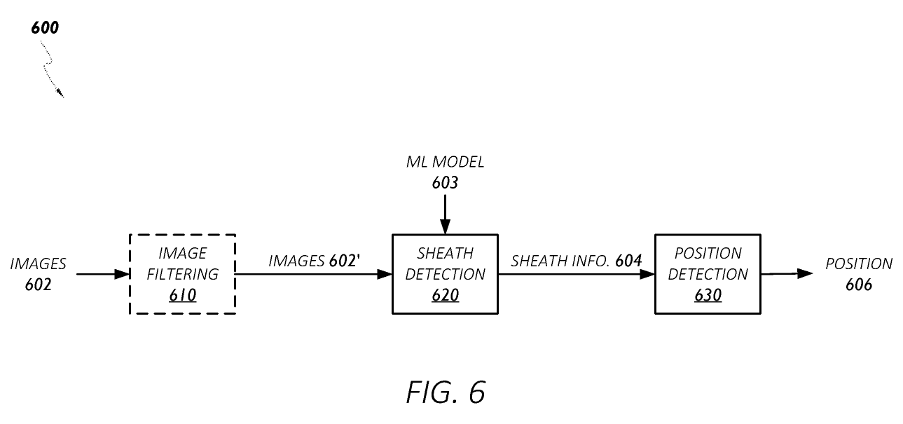

FIG. 6 shows a block diagram of an example system 600 for determining the position of a medical instrument relative to an access sheath, according to some implementations. In some implementations, system 600 may be implemented by a controller for a medical system (such as the medical system 100 of FIG. 1). With reference for example to FIG. 2, the system 600 may include the control circuitry 251 of the control system 50 and/or the control circuitry 211 of the robotic system 10.

The system 600 is configured to receive images 602 (or video) captured by a camera disposed on an elongate shaft of a medical instrument (such as the shaft 40 of FIGS. 1 and 3–5) and determine a position 606 of the distal tip of the shaft relative to the distal end (or opening) of an access sheath through which the shaft is inserted (such as the sheath 90 of FIGS. 1 and 3–5). The images 602 depict a field-of-view (FOV) associated with the distal tip of the medical instrument at any given time (also referred to as “endoscopic vision”), which can help a user navigate the instrument within an anatomy and/or luminal network. For example, the images 602 may be a stream or sequence of video frames that are continuously captured (in real-time) by the camera. As such, changes in the position of the distal tip result in corresponding changes to the scene depicted by the images 602. Thus, the system 600 may use the images 602 to track the movements of the medical instrument (such as insertion and/or retraction) and detect a presence or absence of the access sheath in the FOV of the distal tip.

The system 600 includes a sheath detection component 620 and a position detection component 630. In some implementations, the system 600 also may include an image filtering component 610 to filter the images 602 provided to the processing pipeline. For example, some images 602 may depict “noisy” scenes that may not be suitable for detecting the access sheath or may negatively impact sheath detection (such as scenes containing bubbles, stones, blood, high reflections, remnants of tissue, and/or other occlusions). The image filtering component 610 is configured to denoise the images 602 and/or remove such noisy images from the sequence of images 602 so that the resulting “filtered” images 602’ have at least a threshold level of image quality. For example, the image filtering component 610 may classify each image 602 as “good” or “bad” using one or more image processing techniques and pass only the “good” images (as the filtered images 602’) to the sheath detection component 620. Example suitable image processing techniques include segmentation, machine learning, and statistical analysis, among other examples.

In some implementations, the image filtering component 610 may perform the classification using a machine learning model trained to classify images as “good” or “bad” based on other images previously captured by the same (or similar) camera while performing similar medical procedures. For example, the images used for training may be manually labeled as “good” or “bad” based on whether they depict a relatively clear visual field. In some other implementations, the image filtering component 610 may perform the classification based on statistical analysis. For example, the image filtering component 610 may be configured to track blurriness in the images, or an equivalent histogram distribution, to measure redness and/or color variation. The image filtering component 610 may further be configured to classify each of the images 602 based on the detected levels of redness and/or color variations. For example, images containing high levels of redness and/or low levels of color variation may be classified as “good” whereas images containing low levels of redness and/or high levels of color variation may be classified as “bad.”

The sheath detection component 620 is configured to analyze the images 602’ (or 602) using one or more image processing techniques to identify an inner surface of the access sheath and/or one or more features associated with the anatomy (such as anatomical occlusions) in the FOV of the camera. More specifically, the sheath detection component 620 is configured to output sheath information 604 indicating a presence (or absence) of the access sheath in each image. Example suitable image processing techniques include segmentation, machine learning, and statistical analysis, among other examples. In some implementations, the sheath information 604 may include a classification or label indicating whether the access sheath is present (or absent) in each image. In some other implementations, the sheath information 604 may include one or more bounding boxes indicating the location(s) of the access sheath in each image. Still further, in some implementations, the sheath information 604 may include a segmentation mask indicating, for each pixel of a given image, whether the pixel depicts a respective portion of the access sheath. In some aspects, the sheath detection component 620 may infer the segmentation mask from each image using a machine learning (ML) model 603.

The position detection component 630 is configured to determine the position 606 of the distal tip of the medical instrument relative to the access sheath based, at least in part, on the sheath information 604 associated with the series of images 602’ (or 602). More specifically, the position detection component 630 may determine whether the instrument tip has moved into or out of the access sheath based on the sheath information 604 for the series of images 602’ (or 602). For example, images captured from the distal tip of the instrument may depict the distal end (or opening) of the access sheath when the instrument tip is disposed within the sheath. On the other hand, images captured from the distal tip of the instrument may not depict the distal end (or opening) of the access sheath when the instrument tip is positioned outside the sheath. Thus, any changes in the presence (or absence) of the access sheath indicated by the sheath information 604 over time may coincide with ingress of the instrument tip into the distal opening of the access sheath (such as where the access sheath is absent in a first image but present in a second image captured after the first image) or egress of the instrument tip out from the distal opening of the access sheath (such as where the access sheath is present in a first image but absent in a second image captured after the first image).

As described with reference to FIGS. 4 and 5, the relative position 606 of the distal tip 42 can be used to control the speed of insertion and/or retraction of the shaft 40 by the medical system. For example, when the relative position 606 indicates ingress of the distal tip 42 into the distal end 93 of the sheath 90 or egress of the distal tip 42 out from the distal end 93 of the sheath 90, the medical system can automatically confirm that the distal tip 42 is located within the sheath-entry confirmation zone Z 0. In other words, the medical system can proceed to increase or decrease the rate of insertion and/or retraction of the shaft 40 without any user input or manual confirmation of the sheath tip 93. This systematic approach to determining the relative position 606 of the distal tip 42 eliminates variability in sheath tip confirmation by the system 600. This allows the medical system to detect, with greater confidence, when the distal tip 42 is positioned inside or outside the sheath 90. As a result, the sheath-entry confirmation zone Z 0 and/or buffer zones Z B1 and Z B2 can be reduced or eliminated. In some implementations, the medical system may further display an indication of the relative position 606 of the distal tip 42 on a user interface (such as the display 56 of FIGS. 1 and 2) so that the user can intervene or assume control of the robotic system 400 if the detected position 606 is incorrect.

In the example of FIG. 6, the position information 606 is described as indicating ingress or egress of the distal tip 42 of the shaft 40 relative to the distal end 93 of the sheath 90. However, other information can also be extracted from the images 602’ (or 602). For example, in some implementations, the sheath detection component 620 may be configured to detect rings or other markings on the inner surface of the access sheath and determine a distance traversed by the instrument based on changes to such markings over time. The position detection component 630 may use such information to determine a relationship between motor engagements and the actual distance traveled by the instrument (such as for online backlash detection). “Backlash” refers to gaps or slack between various gears of the robotic system that are used drive the shaft 40. Such gaps or slack can cause a delay, or “dead zone,” in the movement of the shaft 40 when the direction of axial movement is reversed. By detecting the backlash in real-time (or “online”), the medical system can dynamically eliminate minor slippages and/or prevent significant service loop accumulation when driving the medical instrument (such as based on visual servoing).

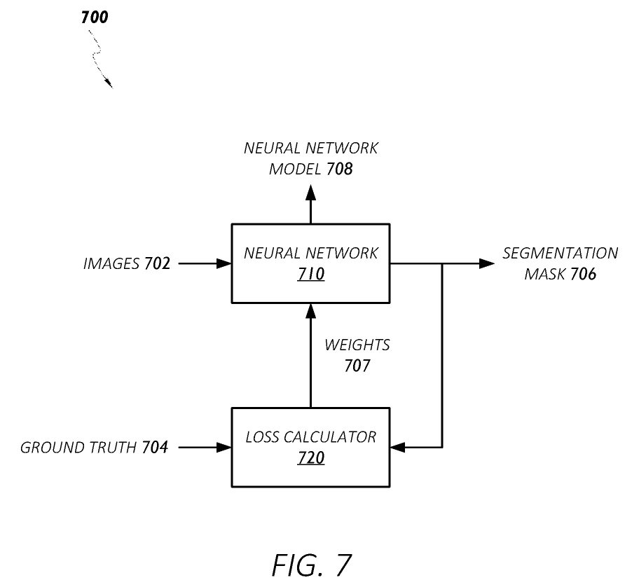

FIG. 7 shows a block diagram of an example machine learning system 700, according to some implementations. The machine learning system 700 is configured to produce a neural network model 708 based, at least in part, on images 702 captured by a camera disposed on the distal tip of an elongate medical instrument (such as the distal tip 42 of FIGS. 1 and 3–5) that is at least partially inserted within an access sheath (such as the sheath 90 of FIGS. 1 and 3–5). In some implementations, the neural network model 708 may be one example of the ML model 603 of FIG. 6. More specifically, the neural network model 708 can be trained to infer a respective segmentation mask 706 (such as a binary mask) for each image 702 indicating which pixels of the image 702 depict an inner surface of the access sheath and which pixels of the image 702 do not depict the inner surface of the sheath. In other words, the segmentation mask 706 delineates the access sheath (if present) from the remainder of the image 702.

The images 702 may include snippets of video recorded during previous medical procedures. More specifically, the images 702 may include a large volume of snippets depicting various complexities that may be present in the surgical environment. Example complexities include visual obstructions (such as blood, tissue remnants, bubbles, stones, and/or reflections) in the FOV of the camera that can obstruct or occlude the access sheath; variability in image quality (such as due to camera focus, lighting conditions, and/or noise); and dynamic scene changes (such as rapid changes to the scene depicted by the images due to movements of the instrument). For example, each image 702 may be reviewed and annotated by a user to delineate the access sheath (if present) from the remainder of the image 702. The annotations associated with each image 702 may be used as ground truth 704 for training the neural network model 708.

The machine learning system 700 includes a neural network 710 and a loss calculator 720. The machine learning system 700 is configured to “train” the neural network 710 to classify each pixel of an image 702 as depicting the access sheath or not depicting the access sheath and aggregate the per-pixel classifications into a respective segmentation mask 706 for the image 702. Deep learning is a particular form of machine learning in which the inferencing and training phases are performed over multiple layers. Deep learning architectures are often referred to as “artificial neural networks” due to the manner in which information is processed (similar to a biological nervous system). For example, each layer of an artificial neural network may be composed of one or more “neurons.” Each layer of neurons may perform a different transformation on the output data from a preceding layer so that the final output of the neural network results in the desired inferences. The set of transformations associated with the various layers of the network is referred to as a “neural network model.” Example suitable neural network architectures include convolutional neural networks (CNNs), recurrent neural networks (RNN), and long short-term memory (LSTM) networks, among other examples.

The neural network 710 receives the images 702, as input, and attempts to learn the ground truth 704 (or annotations) associated with each image 702. For example, the neural network 710 may form a network of connections across multiple layers of artificial neurons that begin with the image 702 and lead to a segmentation mask 706. The connections are weighted to result in a segmentation mask 706 that closely resembles the ground truth 704. In some aspects, the training may be performed over multiple iterations. In each iteration, the neural network 710 produces a segmentation mask 706 based on weighted connections across the layers of artificial neurons, and the loss calculator 720 updates the weights 707 associated with the connections based on an amount of loss (or error) between the segmentation mask 706 and the ground truth 704. The neural network 710 may output the weighted connections as the neural network model 708 when certain convergence criteria are met (such as when the loss falls below a threshold level or when a predetermined number of training iterations have been performed).

As described with reference to FIG. 6, the resulting neural network model 708 can be used to detect a presence or absence of the access sheath in real-time images captured by a camera disposed on the distal tip of a medical instrument during a medical procedure. This allows the system 600 to determine a position 606 of the instrument tip relative to the access sheath and dynamically control or adjust a speed of insertion and/or retraction of the instrument based on the relative position 606 (such as described with reference to FIGS. 4 and 5).

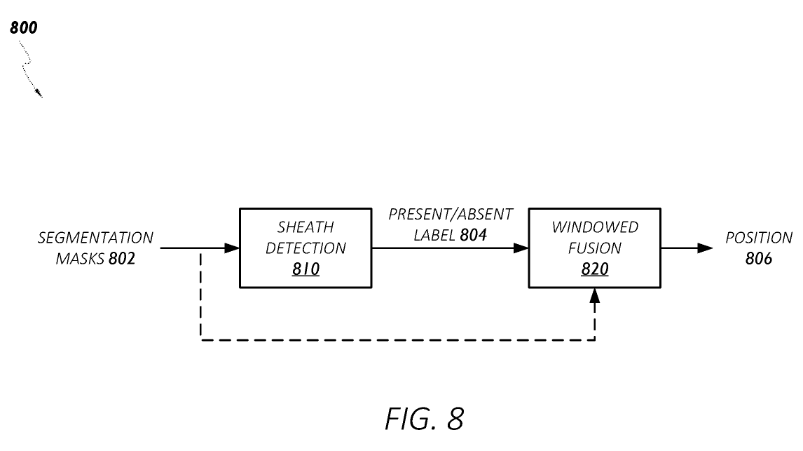

FIG. 8 shows a block diagram of an example instrument position detection system 800, according to some implementations. In some implementations, the instrument position detection system 800 may be one example of the position detection component 630 of FIG. 6. More specifically, the instrument position detection system 800 is configured to determine a position 806 of the distal tip of an elongate instrument (such as the distal tip 42 of FIGS. 1 and 3–5) relative to an access sheath (such as the sheath 90) based on a series of segmentation masks 802 associated with a series of images, respectively, captured by a camera disposed on the distal tip of the instrument (such as the images 602’ or 602 of FIG. 6).

In some implementations, the segmentation masks 802 may be inferred from the images using a machine learning model trained to classify each pixel of an image as depicting the access sheath or not depicting the access sheath (such as the ML model 603 of FIG. 6 or the neural network model 708 of FIG. 7). More specifically, each segmentation mask 802 may indicate, for each pixel of the corresponding image, whether the pixel depicts the access sheath or does not depict the access sheath. In other words, each segmentation mask 802 indicates which (if any) pixels of a given image depict a portion of the access sheath.

The instrument position detection system 800 includes a sheath detection component 810 and a windowed fusion component 820. The sheath detection component 810 is configured to determine whether the access sheath is present or absent in a given image based on the segmentation mask 802 associated with the image. More specifically, the sheath detection component 810 may output a binary classification label 804 indicating whether the sheath is present or absent based on the segmentation mask 802. In some implementations, the sheath detection component 810 may count a number (N) of pixels depicting the access sheath in each segmentation mask 802 and compare the number N to a threshold amount (or percentage). For example, if the number N of pixels depicting the access sheath is greater than or equal to the threshold amount, the sheath detection component 810 may label 804 the segmentation mask 802 as depicting a presence of the access sheath. On the other hand, if the number N of pixels depicting the access sheath is less than the threshold amount, the sheath detection component 810 may label 804 the segmentation mask 802 as depicting an absence of the access sheath.

The windowed fusion component 820 is configured to buffer or aggregate the labels 804 over a period of time (corresponding to a series of images or segmentation masks 802) and determine the relative position 806 of the instrument tip based on the aggregated labels 804. As described with reference to FIG. 6, any changes in the presence (or absence) of the access sheath indicated by the label 804 over time may coincide with ingress of the instrument tip into the distal opening of the access sheath or egress of the instrument tip out from the distal opening of the access sheath. In some implementations, the windowed fusion component 820 may output position information 806 indicating ingress of the instrument tip (into the access sheath) in response to receiving one or more labels 804 indicating the sheath is absent followed by one or more labels 804 indicating the sheath is present. In some other implementations, the windowed fusion component 820 may output position information 806 indicating egress of the instrument tip (out of the access sheath) in response to receiving one or more labels 804 indicating the sheath is present followed by one or more labels 804 indicating the sheath is absent.

As described with reference to FIG. 7, various complexities in the surgical environment (such as visual obstructions, variability in image quality, and/or dynamic scene changes) can interfere with the ability of a machine learning model to detect the access sheath from images of the environment. As a result, some segmentation masks 802 may include false-positive classifications of pixels depicting the access sheath (or not depicting the access sheath), which can lead to incorrect labels 804 indicating a presence or absence of the sheath. In some implementations, the windowed fusion component 820 may further track one or more changes to the segmentation masks 802 over time to determine a confidence value for each label 804. For example, sudden and/or significant changes to the shape or size of the segmentation masks 803 may be attributed to false-positive classifications by the machine learning model. Thus, the windowed fusion component 820 may assign low confidence values to any labels 804 associated with segmentation masks 802 in which a sudden change is detected. In some implementations, the windowed fusion component 820 may infer the confidence values from the segmentation masks 802 using a temporal tracking machine learning model. Example suitable machine learning architectures include LSTMs and transformers, among other examples.

Aspects of the present disclosure recognize that any changes to the segmentation masks 802 should be consistent with the speed and direction of axial movement by the medical instrument. Thus, in some implementations, the windowed fusion component 820 may further determine the confidence values for the labels 804 based, at least in part, on user inputs or robotic commands (not shown for simplicity) for controlling movement of the instrument. For example, if the access sheath appears to be disappearing from the segmentation masks 802 over time, but the user input indicates that the medical instrument is being retracted, the windowed fusion component 820 may detect a discrepancy between the segmentation masks 802 and the associated user inputs.

Aspects of the present disclosure further recognize that the distal tips of some medical instruments may include additional sensors (such as pressure sensors) that can aid in detecting when the instrument tip crosses the distal opening of the access sheath. For example, the pressure sensor may register a significant jump in pressure when the instrument tip egresses out of the distal opening of the sheath (and presses against the anatomy). Thus, in some other implementations, the windowed fusion component 820 may further determine the confidence values for the labels 804 based, at least in part, on additional sensor data received from the distal tip of the instrument. For example, if the sheath appears to be expanding in the segmentation masks 802 over time, which coincides with a sudden spike in pressure detected by a pressure sensor at the distal tip of the instrument, the windowed fusion component 820 may detect a discrepancy between the segmentation masks 802 and the associated sensor data.

In some implementations, the windowed fusion component 820 may assign relatively low confidence values to labels 804 associated with any segmentation masks 802 for which discrepancies are detected or otherwise appear to be inconsistent with received user inputs and/or other sensor data. In some implementations, the windowed fusion component 820 may ignore any labels 804 having low confidence values in determining the relative position 806 of the instrument tip. In some other implementations, the windowed fusion component 820 may output the confidence values together with the position information 806 (such as for display on a user interface and/or display device).

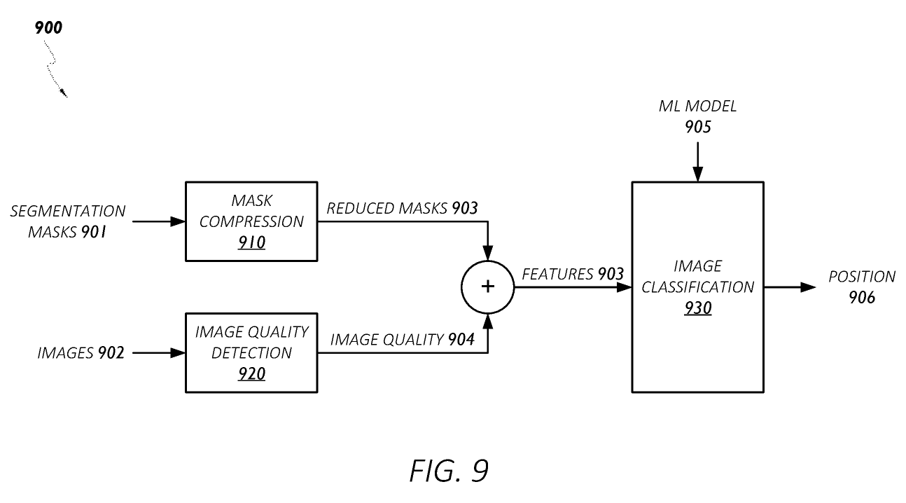

FIG. 9 shows another block diagram of an example instrument position detection system 900, according to some implementations. In some implementations, the instrument position detection system 900 may be one example of the position detection component 630 of FIG. 6. More specifically, the instrument position detection system 900 is configured to determine a position 906 of the distal tip of an elongate instrument (such as the distal tip 42 of FIGS. 1 and 3–5) relative to an access sheath (such as the sheath 90) based on a series of segmentation masks 901 associated with a series of images 902, respectively, captured by a camera disposed on the distal tip of the instrument (such as the images 602’ or 602 of FIG. 6).

In some implementations, the segmentation masks 901 may be inferred from the images 902 using a machine learning model trained to classify each pixel of an image 902 as depicting the access sheath or not depicting the access sheath (such as the ML model 603 of FIG. 6 or the neural network model 708 of FIG. 7). More specifically, each segmentation mask 901 may indicate, for each pixel of the corresponding image 902, whether the pixel depicts the access sheath or does not depict the access sheath. In other words, each segmentation mask 901 indicates which (if any) pixels of the respective image 902 depict a portion of the access sheath.

The instrument position detection system 900 includes a mask compression component 910, an image quality detection component 920, and an image classification component 930. The mask compression component 910 is configured to reduce the size and/or granularity of each segmentation mask 901. For example, the mask compression component 910 may produce a “reduced” mask 903 through compression or max-pooling of the per-pixel classifications in a corresponding segmentation mask 901. As a result, the reduced mask 903 may capture lower-level features of the access sheath compared to the segmentation mask 901.

The image quality detection component 920 is configured to determine an image quality 904 of each of the images 902. For example, images 902 depicting noisy scenes (such as scenes containing bubbles, stones, blood, high reflections, remnants of tissue, and/or other occlusions) may be assigned lower image quality values 904, whereas images 902 depicting less noisy scenes may be assigned higher image quality values 904. The image quality value 904 for each image 902 is combined or concatenated with the reduced mask 903 associated with the image 902 to create a respective multidimensional feature 903. In some implementations, the multidimensional feature 903 may include one or more additional features, such as instrument insertion or retraction values and/or various pressure sensor values (not shown for simplicity).

The image classification component 930 is configured to analyze the features 903 for changes in the presence (or absence) of the access sheath over time and further classify each feature 903 based on any detected changes in the presence (or absence) of the sheath. More specifically, the image classification component 930 may determine a respective classification for each feature 903 indicating a position 906 of the instrument tip relative to the access sheath. In some implementations, the image classification component 930 may implement a temporal tracking ML model 905 trained to perform the classification on a time-series multidimensional feature set. Example suitable machine learning architectures include LSTMs and transformers, among other examples. In contrast with the relative position 806 of FIG. 8, the relative position 906 is a frame-level prediction indicating ingress or egress of the instrument tip per image 902.

As described with reference to FIGS. 6, 8 and 9, the position information 606, 806, and 906 can be used by a controller for a robotic system to control the speed of insertion and/or retraction of a medical instrument during a medical procedure. Aspects of the present disclosure further recognize that the position information 606, 806, and 906 can be used for various other purposes in addition to controlling the speed of insertion of the medical instrument. For example, in some implementations, a medical system may calculate the actual length of the access sheath by synthesizing the state of the robotic system, arm positioning, length of the elongate medical instrument, and the position of the instrument tip relative to the sheath (such as when the position information 606, 806, or 906 indicates ingress or egress of the instrument tip). In some other implementations, the medical system may further leverage the position information 606, 806, or 906 to detect an amount of slack in the instrument shaft and mitigate the service loop as necessary.

With reference for example to FIG. 4, some implementations of fast retraction of a scope or other instrument involve the translation of a robotic end effector 6a (e.g., distal end effector of a robotic arm) coupled to an instrument handle/base 31 in a direction generally parallel to and/or in-line with a virtual rail 401 that is aligned with an axis of the access sheath 90, instrument/scope feeder channel 39, and/or alignment between the end effector 6a and the end effector 8b that is associated with the instrument driver/feeder 11. That is, the robotic movement or translation of the arm 12a and/or end effector 6a may generally be in a direction or dimension 402 during fast retraction and/or insertion of the shaft 40, wherein such movement may advantageously facilitate relatively fast retraction/insertion and/or reduce the size of the service loop 49 formed in the shaft 40 and/or the radii of bends formed in the shaft 40 associated with the service loop 49.

Translation of the end effector 6a in the proximal direction during instrument retraction can advantageously increase the distance D b between the instrument driver/feeder 11 and the instrument base 31, thereby reducing the length of shaft that is inclined to bunch-up to form the service loop 49 relative to implementations in which no proximal instrument handle or base translation occurs. Where the proximal translation of the end effector 6a is constrained to the linear dimension 402 and/or 401, which is referred to herein as the ‘x’ dimension in some contexts for convenience, the increase in the distance D b between the instrument driver 11 and the base 31 of the shaft 40 resulting from translation of the end effector 6a may generally be equal to the translation distance in the x-dimension. Such translation in the x-dimension may be limited by mechanical constraints of the end effector 6a and/or the robotic arm or system. Therefore, the amount of strain relief provided by linear translation of the instrument handle 31 (in the x-direction) as shown in FIG. 4 also may be limited by such mechanical constraints.

As described above, the service loop 49 may form when the length of the shaft 40 disposed between the instrument driver/feeder 11 and the instrument handle/base 31 increases due to retraction of the instrument driver 11 at a speed that is greater than a retraction translation speed of the end effector 6a. As shown, the service loop 49 may form as a U-bend including base bends 99a,99c on either side of an apex bend 99b. Generally, the greater the length of shaft 40 forming the service loop 49, the greater the transverse deflection d t of the service loop 49. As the deflection d t of the service loop 49 increases, the radii of curvature of the bends 99 formed in the shaft 40 are reduced, thereby resulting in relatively sharper/tighter bends in the shaft 40.

It may be desirable to avoid the formation of relatively tight/sharp bends in the shaft 40 to avoid damage to the instrument due to mechanical stress. FIG. 4 shows the service loop 49 forming three bends 99, including a first bend 99a between the instrument driver/feeder 11 and the apex 404 of the service loop 49, wherein such bend 90a is illustrated as having a radius of curvature r 1, which may be relatively short in some implementations in which the feed-roller retraction speed relative to the proximal retraction of the end effector 6a is relatively high. The apex bend 99b is shown as having a radius of curvature r 2, whereas the third bend between the base 62 of the shaft 40 and the apex 404 of the service loop 49 has a radius of curvature r 3.

Generally, the difference between the retraction speed of the instrument driver 11 and the linear translation speed of the end effector 6a along the virtual rail 401 can cause the shaft 40 to form the service loop 49, wherein the severity of mechanical stress/strain imposed by the various bends of the service loop 49 may be dependent at least in part on the proximal translation distance traversed during the retraction feeding of the instrument driver 11. By detecting the amount of slack in the shaft 40 at any given time using the position information 606, 806, or 906 of FIGS. 6, 8, and 9, respectively, the controller for the robotic system 400 can take appropriate corrective action to reduce the radii of the bends in the service loop 49, for example, through transverse and/or proximal instrument base translation during instrument driver retraction.

Although FIG. 4 shows linear translation of the shaft 40 in the x-dimension along the virtual rail 401, in some implementations, instrument retraction solutions associated with the present disclosure can involve end effector translation in directions or dimensions that are transversed or angled relative to the virtual rail 401. For example, instrument base translation in the illustrated ‘y’ and/or ‘z’ dimensions, either separately or in combination with each other and/or in combination with translation in the ‘x’ dimension, can increase the distance D b between the instrument base 31 and the instrument driver 11 and/or produce angular or orientational positions of various portions of the shaft 40 that reduce bend curvature radii in one or more areas associated with a service loop.

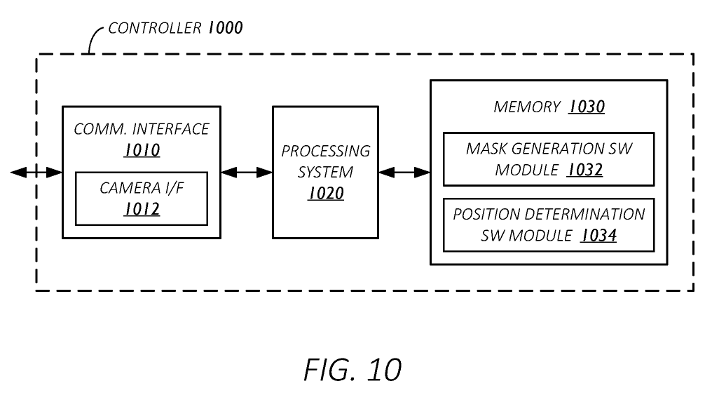

FIG. 10 shows a block diagram of an example controller 1000 for a medical system, according to some implementations. In some implementations, the controller 1000 may be one example of the system 600 of FIG. 6 or any of the control circuitry 251 and/or 211 of FIG. 2. More specifically, the controller 1000 is configured to determine relative instrument positions based on images captured by a camera disposed on the distal tip of an elongate shaft.

The controller 1000 includes a communication interface 1010, a processing system 1020, and a memory 1030. The communication interface 1010 is configured to communicate with one or more components of the medical system. More specifically, the communication interface 1010 includes a camera interface (I/F) 1012 for communicating with the camera on the distal tip of the instrument shaft (such as the distal tip 42 of FIGS. 1 and 3–5). In some implementations, the camera I/F 1012 may obtain a series of images captured by the camera disposed on the distal tip of the medical instrument while the instrument is at least partially inserted through an access sheath.

The memory 1030 may include a non-transitory computer-readable medium (including one or more nonvolatile memory elements, such as EPROM, EEPROM, Flash memory, or a hard drive, among other examples) that may store the following software (SW) modules: a mask generation SW module 1032 to infer a respective segmentation mask from each image in the series of images based on a machine learning model trained to classify each pixel of the image as depicting the access sheath or not depicting the access sheath, where the segmentation mask indicates how many pixels of the image are classified as depicting the access sheath; and a position determination SW module 1034 to determine a position of the distal tip of the medical instrument relative to the access sheath based at least in part on the segmentation masks for the series of images.

The processing system 1020 may include any suitable one or more processors capable of executing scripts or instructions of one or more software programs stored in the controller 1000 (such as in the memory 1030). For example, the processing system 1020 may execute the mask generation SW module 1032 to infer a respective segmentation mask from each image in the series of images based on a machine learning model trained to classify each pixel of the image as depicting the access sheath or not depicting the access sheath, where the segmentation mask indicates how many pixels of the image are classified as depicting the access sheath. The processing system 1020 also may execute the position determination SW module 1034 to determine a position of the distal tip of the medical instrument relative to the access sheath based at least in part on the segmentation masks for the series of images.

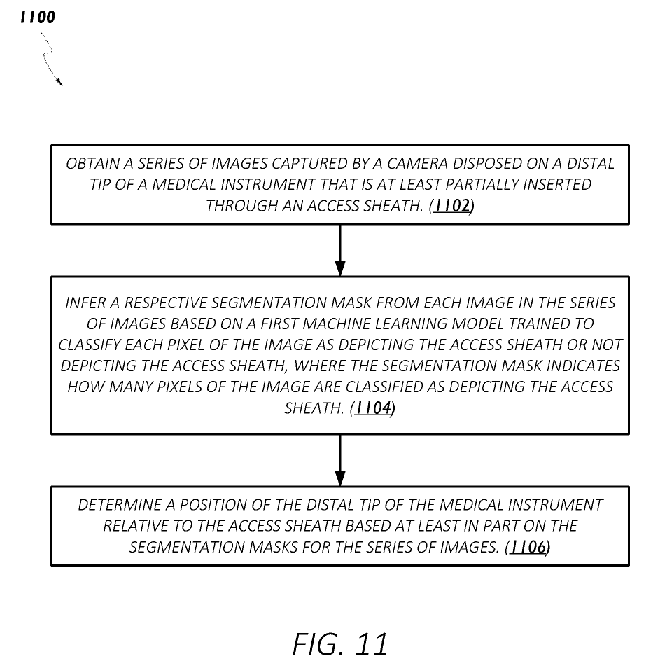

FIG. 11 shows an illustrative flowchart depicting an example operation 1100 for determining relative instrument positions, according to some implementations. In some implementations, the example operation 1100 may be performed by a controller for a medical system such as the controller 1000 of FIG. 10 or the system 600 of FIG. 6.

The controller obtains a series of images captured by a camera disposed on a distal tip of a medical instrument that is at least partially inserted through an access sheath (1102). In some aspects, the obtaining of the series of images may include receiving a plurality of images captured in sequential order by the camera, determining a quality of each image of the plurality of images, and filtering the plurality of images based on the quality of each image so that the series of images includes only the filtered plurality of images arranged according to the sequential order by which they are captured. The controller also infers a respective segmentation mask from each image in the series of images based on a first machine learning model trained to classify each pixel of the image as depicting the access sheath or not depicting the access sheath, where the segmentation mask indicates how many pixels of the image are classified as depicting the access sheath (1104). The controller further determines a position of the distal tip of the medical instrument relative to the access sheath based at least in part on the segmentation masks for the series of images (1106).

In some aspects, the position of the distal tip of the medical instrument relative to the access sheath may be inferred based on a second machine learning model trained to predict whether the distal tip of the medical instrument is positioned within the access sheath or outside the access sheath based on the segmentation masks for the series of images. In some other aspects, the controller may classify one or more first images in the series of images as depicting a presence of the access sheath based on the segmentation mask for each of the one or more first images indicating that at least a threshold number of pixels are classified as depicting the access sheath; and classify one or more second images in the series of images as depicting an absence of the access sheath based on the segmentation mask for each of the one or more second images indicating that less than the threshold number of pixels are classified as depicting the access sheath. In some implementations, the controller may determine a confidence value associated with the classification for each of the one or more first images and each of the one or more second images based at least in part on the segmentation masks for the series of images.

In some implementations, the determining of the positions of the distal tip of the medical instrument may include determining that the one or more first images occur earlier in the series than the one or more second images, and detecting egress of the distal tip of the medical instrument from a distal opening of the access sheath responsive to determining that the one or more first images occur earlier in the series than the one or more second images. In some other implementations, the determining of the positions of the distal tip of the medical instrument may include determining that the one or more first images occur later in the series than the one or more second images, and detecting ingress of the distal tip of the medical instrument into a distal opening of the access sheath responsive to determining that the one or more first images occur later in the series than the one or more second images.

In some aspects, the controller may further track one or more features of the access sheath across two or more images in the series of images and determine a speed or distance of travel by the medical instrument based on tracking the one or more features. In some other aspects, the controller may further determine a length of the access sheath based at least in part on the determined position of the distal tip of the medical instrument relative to the access sheath and a known length of the medical instrument. Still further, in some aspects, the controller may further determine an amount of slack in an elongate shaft of the medical instrument based at least in part on the determined position of the distal tip of the medical instrument relative to the access sheath and a known length of the medical instrument.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method for determining relative instrument positions, comprising:
obtaining a series of images captured by a camera disposed on a distal tip of a medical instrument that is at least partially inserted through an access sheath;
inferring a respective segmentation mask from each image in the series of images based on a first machine learning model trained to classify each pixel of the image as depicting the access sheath or not depicting the access sheath, the segmentation mask indicating how many pixels of the image are classified as depicting the access sheath; and
determining a position of the distal tip of the medical instrument relative to the access sheath based at least in part on the segmentation masks for the series of images.

2. The method of claim 1, wherein the position of the distal tip of the medical instrument relative to the access sheath is inferred based on a second machine learning model trained to predict whether the distal tip of the medical instrument is positioned within the access sheath or outside the access sheath based on the segmentation masks for the series of images.

3. The method of claim 1, further comprising:
classifying one or more first images in the series of images as depicting a presence of the access sheath based on the segmentation mask for each of the one or more first images indicating that at least a threshold number of pixels are classified as depicting the access sheath; and
classifying one or more second images in the series of images as depicting an absence of the access sheath based on the segmentation mask for each of the one or more second images indicating that less than the threshold number of pixels are classified as depicting the access sheath.

4. The method of claim 3, further comprising:
determining a confidence value associated with the classification for each of the one or more first images and each of the one or more second images based at least in part on the segmentation masks for the series of images.

5. The method of claim 3, wherein the determining of the position of the distal tip of the medical instrument comprises:
determining that the one or more first images occur earlier in the series than the one or more second images; and
detecting egress of the distal tip of the medical instrument from a distal opening of the access sheath responsive to determining that the one or more first images occur earlier in the series than the one or more second images.

6. The method of claim 3, wherein the determining of the position of the distal tip of the medical instrument comprises:
determining that the one or more first images occur later in the series than the one or more second images; and
detecting ingress of the distal tip of the medical instrument into a distal opening of the access sheath responsive to determining that the one or more first images occur later in the series than the one or more second images.

7. The method of claim 1, wherein the obtaining of the series of images comprises:
receiving a plurality of images captured in sequential order by the camera;
determining a quality of each image of the plurality of images; and
filtering the plurality of images based on the quality of each image so that the series of images includes only the filtered plurality of images arranged according to the sequential order by which they are captured.

8. The method of claim 1, further comprising:
tracking one or more features of the access sheath across two or more images in the series of images; and
determining a speed or distance of travel by the medical instrument based on tracking the one or more features.

9. The method of claim 1, further comprising:
determining a length of the access sheath based at least in part on the determined position of the distal tip of the medical instrument relative to the access sheath and a known length of the medical instrument.

10. The method of claim 1, further comprising:
determining an amount of slack in an elongate shaft of the medical instrument based at least in part on the determined position of the distal tip of the medical instrument relative to the access sheath and a known length of the medical instrument.

11. A. controller for a medical system comprising:
a processing system;
a memory storing instructions that, when executed by the processing system, cause the controller to:
obtain a series of images captured by a camera disposed on a distal tip of a medical instrument that is at least partially inserted through an access sheath;
infer a respective segmentation mask from each image in the series of images based on a first machine learning model trained to classify each pixel of the image as depicting the access sheath or not depicting the access sheath, the segmentation mask indicating how many pixels of the image are classified as depicting the access sheath; and
determine a position of the distal tip of the medical instrument relative to the access sheath based at least in part on the segmentation masks for the series of images.

12. The controller of claim 11, wherein the position of the distal tip of the medical instrument relative to the access sheath is inferred based on a second machine learning model trained to predict whether the distal tip of the medical instrument is positioned within the access sheath or outside the access sheath based on the segmentation masks for the series of images.

13. The controller of claim 11, wherein execution of the instructions further causes the controller to:
classify one or more first images in the series of images as depicting a presence of the access sheath based on the segmentation mask for each of the one or more first images indicating that at least a threshold number of pixels are classified as depicting the access sheath; and
classify one or more second images in the series of images as depicting an absence of the access sheath based on the segmentation mask for each of the one or more second images indicating that less than the threshold number of pixels are classified as depicting the access sheath.

14. The controller of claim 13, wherein execution of the instructions further causes the controller to:
determine a confidence value associated with the classification for each of the one or more first images and each of the one or more second images based at least in part on the segmentation masks for the series of images.

15. The controller of claim 13, wherein the determining of the position of the distal tip of the medical instrument comprises:
determining that the one or more first images occur earlier in the series than the one or more second images; and
detecting egress of the distal tip of the medical instrument from a distal opening of the access sheath responsive to determining that the one or more first images occur earlier in the series than the one or more second images.

16. The controller of claim 13, wherein the determining of the position of the distal tip of the medical instrument comprises:
determining that the one or more first images occur later in the series than the one or more second images; and
detecting ingress of the distal tip of the medical instrument into a distal opening of the access sheath responsive to determining that the one or more first images occur later in the series than the one or more second images.

17. The controller of claim 11, wherein the obtaining of the series of images comprises:
receiving a plurality of images captured in sequential order by the camera;
determining a quality of each image of the plurality of images; and
filtering the plurality of images based on the quality of each image so that the series of images includes only the filtered plurality of images arranged according to the sequential order by which they are captured.

18. The controller of claim 11, wherein execution of the instructions further causes the controller to:
track one or more features of the access sheath across two or more images in the series of images; and
determine a speed or distance of travel by the medical instrument based on tracking the one or more features.

19. The controller of claim 11, wherein execution of the instructions further causes the controller to:
determine a length of the access sheath based at least in part on the determined position of the distal tip of the medical instrument relative to the access sheath and a known length of the medical instrument.

20. The controller of claim 12, wherein execution of the instructions further causes the controller to:
determine an amount of slack in an elongate shaft of the medical instrument based at least in part on the determined position of the distal tip of the medical instrument relative to the access sheath and a known length of the medical instrument.
